(19) United States
(12) Patent Application Publication
(10) Pub. No.: US 2004/0137456 A1
(43) Pub. Date: Jul. 15, 2004

(54) Method for identifying and characterizing individual dna molecules

(76) Inventors: Hiroki Yokota, Carmel, IN (US); Hui Bin Sun, Indianapolis, IN (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/473,787

(22) PCT Filed: Apr. 4, 2002
(86) PCT No.: PCT/US02/10696

(51) Int. Cl. C12Q 1/68

(52) U.S. Cl. 435/6

(57) ABSTRACT

Methods are provided that facilitate the identification and characterization of individual nucleic acid molecules.

This application claims priority under 35 U.S.C. §119(e) to U.S. Provisional Application 60/281,469 filed Apr. 4, 2001, the entire disclosure of which is incorporated by reference herein.

### FIELD OF THE INVENTION

This invention relates to the fields of molecular genetics and molecular diagnostic assays. More specifically, the present invention provides methods that facilitate the identification and characterization of nucleic acid molecules present in biological samples.

### BACKGROUND OF THE INVENTION

Several publications and patent documents are referenced in this application by numerals in parentheses in order to more fully describe the state of the art to which this invention pertains. Full citations for these references are found at the end of the specification. The disclosure of each of these publications is incorporated by reference herein.

The near completion of the human genome project provides many new opportunities to more accurately study genetic alterations, mutations and DNA polymorphisms in humans (1,2). DNA sequence alterations are of particular interest because they often result in the production of defective proteins giving rise to genetic disorders. An understanding of the relationship between sequence variations and disease is highly desirable. Such knowledge facilitates the development of beneficial therapeutic agents for the treatment of genetic disorders.

Abasic sites, or apurinic/apyrimidinic sites (AP sites), are one of the most commonly identified DNA sequence alterations. AP sites are formed when a nucleotide base, such as Adenine (A), Cytosine (C), Guanine (G), or Thymine (T) is removed from a polynucleotide strand (3, 4). Formation of AP sites occurs naturally in DNA by spontaneous depurination or deamination or by chemically-induced hydrolysis of the N-glcosylic bond, followed by the removal of the base from the polynucleotide strand by the enzymatic activity of DNA glycosylases. It has been estimated that nearly 10,000 AP sites arise per mammalian genome per day under normal physiological conditions.

AP sites are non-coding lesions on the polynucleotide strand and thus represent potentially lethal or mutagenic damages. The removal of a nucleotide base from the polynucleotide strand not only leads to an alteration in the DNA sequence, but may also lead to genetic disorders, carcinogenesis and cell death (5).

Atomic force microscopy (AFM) provides a powerful visualization tool for identifying DNA alterations, including AP sites, on individual DNA molecules (6-8). AFM is a type of scanning probe microscopy that characterizes surface properties, such as topography or friction by a spatially controlled probe. This form of microscopy is often used to capture structural images of biological molecules at nanometer resolution, including nucleic acids, proteins and cell membranes (9-11). Using AFM as opposed to electrophoretic mobility shift assays, DNase footprinting, or microfabricated DNA arrays, allows the investigator to analyze individual nucleic acid molecules of greater length.

Although several methods are presently available for quantifying the level of AP sites from a population of chromosomes or cells, no method is capable of assaying the physical position or the distribution of AP sites on individual DNA molecules (12-15). Such a method would be extremely beneficial for developing new technologies that are capable of rapidly comparing similarities and differences between various individual DNA molecules. This is especially true as many genetic disorders are caused by point mutations.

### SUMMARY OF THE INVENTION

In accordance with the present invention, a method for identifying and characterizing individual nucleic acid molecules is provided. An exemplary method of the invention comprises: (1) forming abasic sites specific for at least one nucleic acid base on an individual nucleic acid molecule, (2) labeling said abasic sites with a detectably labeled molecule, and (3) detecting the nature and location of the detectably labeled abasic sites. The individual nucleic acid molecule may be either DNA or cDNA, and the abasic sites are formed with a glycosylase. Suitable glycosylases for this purpose include without limitation, uracil-DNA glycosylase, 3-methyladenine-DNA glycosylase, AlkA protein, 5-methylcytosine-DNA glycosylase, Fpg protein, and Tag protein. In one embodiment, detection of the labeled abasic sites is performed by atomic force microscopy.

In an additional embodiment of the present invention, the method may be used to advantage to determine the sequence of an individual nucleic acid molecule by creating four nucleic acid sequences, wherein each of said four nucleic acid sequences contains abasic sites corresponding to one of the four particular nucleic acid bases, followed by the determination of the location of each abasic site in each of the four nucleic acid molecules.

In yet another embodiment of the invention, the method may be used to advantage to identify and characterize individual nucleic acid molecules containing abasic sites that are naturally formed in vivo caused by ionizing radiation, mutagenic chemical or spontaneous mutations. The method may also be used to advantage to identify patients at risk for certain genetic disorders. Additionally, the method of the invention may be used to determine the paternity of an individual.

### BRIEF DESCRIPTION OF THE DRAWINGS

FIG. 1 shows a diagram outlining the method used to create AP sites on an individual DNA template. The DNA template was biotinylated at the 5′-end, and the two AP sites were formed by uracil N-glycosylase. The AP sites were then reacted with biotinylated aldehyde-reactive probe (bio-ARP) and subsequently with monomeric Avidin, approximately 16 kDa in mass.

FIGS. 2A-2E show atomic force microscopy height images of avidin, DNA, and Avidin-bound DNA. Height is indicated by a color code with dark (0 nm) and light (3 nm). FIG. 2A shows the monomeric Avidin, 16 kDa in mass. FIG. 2B shows the spin-stretched 250-bp DNA templates without any labeling. The mean and the standard deviation of the end-to-end length was measured as 77±5 nm (N=30). FIGS. 2C, 2D and 2E show the Avidin-DNA complexes formed after incubation with bio-ARP and monomeric Avidin.

FIG. 3 shows a histogram of the distribution of bound Avidins on the stretched DNA templates. The mean and the standard deviation for the two AP sites were measured as 0.28±0.02 (N=29) and 0.35±0.02 (N=29), which is in good agreement with the predicted values of 0.27 and 0.35, respectively.

FIGS. 4A-4C show atomic force microscopy height images of the end-labeled Avidin-DNA complexes. In order to illustrate three-dimensional features, the topographical images are displayed by tilting the mica surface 20 degrees. The white arrow indicates the bound Avidin monomer. FIG. 4A shows the DNA templates end-labeled with Avidin. FIG. 4B shows the end-labeled DNA template with one of the two AP sites labeled with Avidin. FIG. 4C shows the end-labeled DNA template with both of the two AP sites labeled with Avidin.

### DETAILED DESCRIPTION OF THE INVENTION

DNA is a complex biological molecule of finite chemical stability. Its sugar-phosphate backbone can be broken by oxidative stress or ionizing radiation, and its bases can be altered both enzymatically and by mutagens (16-18). Abasic or apurinic/apyrimidinic sites (AP sites) are one of the most prevalent DNA lesions. Such sites arise when a DNA base (A, C, G, or T) is removed from a polynucleotide strand (3,4). Detection of AP sites in conjunction with examination of the genetic integrity of individual DNA molecules facilitates the diagnosis of genetic disorders. Since there are different forms of DNA damage which can vary from one DNA molecule to another, a sensitive and reliable method for detecting AP sites on individual DNA molecules is highly desirable. Several methods are presently available for quantifying the level of AP sites in a population of chromosomes or cells; however, none of the current methods provide means to assess the physical position or distribution of AP sites on individual DNA molecules (13-15).

In accordance with the present invention, a method has been developed which physically locates abasic (or AP) sites on individual nucleic acid molecules and facilitates the rapid characterization and identification of individual nucleic acid molecules using a DNA-binding agent specific for AP sites. This novel method of abasic site DNA labeling comprises the steps of: (a) creating AP sites at specific locations in an individual DNA molecule, (b) labeling the AP sites by marking them with probes specific for the AP sites, and (c) detecting the AP sites using atomic force microscopy or fluorescence microscopy.

The present invention is advantageous over existing methods for several reasons: (1) AP sites are created in a DNA sequence-dependent manner, (2) individual AP sites are labeled and detected on individual DNA molecules, and (3) single DNA molecules are characterized based on the distribution of labeled AP sites using atomic force microscopy or fluorescence microscopy. These unique features facilitate the characterization of individual DNA molecules.

Unlike conventional methods, such as color-coding with in situ hybridization technique or specific cuts with optical mapping techniques, the present invention places many AP sites as landmarks on individual DNA molecules in a DNA-sequence dependent manner. Therefore, the present invention provides an excellent tool for identifying similarities and differences among various DNA samples. Additionally, the present invention may be used to advantage as a tool for identifying genetic alterations which cause genetic disorders, carcinogenesis and cell death.

In an alternative embodiment of the invention, probes or PCR primers may be employed to identify AP sites on individual DNA molecules. Oligonucleotide probes may be used to place landmarks on individual DNA molecules by hybridizing end-labeled oligonucleotide probes to specific locations on the target molecule. Alternatively, PCR may be used to identify AP sites on individual DNA molecules by monitoring the length of PCR fragments. The presence of AP sites on a DNA molecule blocks the polymerization of new DNA fragments during PCR. Thus, shorter DNA fragments are generated from DNA molecules with AP sites than from DNA molecules without AP sites.

The following description sets forth the general procedures involved in practicing the present invention. To the extent that specific materials are mentioned, it is merely for purposes of illustration and is not intended to limit the invention. Unless otherwise specified, general biochemical and molecular biological procedures, such as those set forth in Sambrook et al., Molecular Cloning, Cold Spring Harbor Laboratory (1989) (hereinafter “Sambrook et al.”) or Ausubel et al. (eds) Current Protocols in Molecular Biology, John Wiley & Sons (1997) (hereinafter “Ausubel et al.”) are used.

I. Definitions

The following definitions are provided to facilitate an understanding of the present invention:

“Abasic sites” or “AP sites” are non-coding lesions on a polynucleotide strand where a single DNA base such as A, C, G or T is removed.

“Atomic Force Microscopy” is a type of scanning probe microscopy that characterizes surface properties such as topography or friction by a spatially controlled probe. AFM is a versatile tool for detecting and imaging biomolecules at nanometric i.e., single molecule, resolution. It can be applied to detect DNA, protein or DNA-binding-protein complexes. “Fluorescence Microscopy” is used to visualize specimens that fluoresce or emit light of one color when light of another color shines upon them. Fluorescence occurs either because of naturally occurring fluorescent substances found within a specimen such as chlorophyll or other fluorescing components (autofluorescence) or because the specimens have been coupled with a fluorescent dye.

With reference to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. An “isolated nucleic acid molecule” may also comprise a cDNA molecule or a recombinant nucleic acid molecule.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA molecule of the invention, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., 1989):

Tₘ=81.5° C.+16.6Log [Na+]+0.41(% G+C)−0.63(% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The term “probe” as used herein refers to an oligonucleotide, polynucleotide or DNA molecule, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to a DNA oligonucleotide, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a. polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

The term “specific binding pair” as used herein includes antigen-antibody, receptor-hormone, receptor-ligand, agonist-antagonist, lectin-carbohydrate, nucleic acid (RNA or DNA) hybridizing sequences, Fc receptor or mouse IgG-protein A, avidin-biotin, streptavidin-biotin, amine-reactive agent-amine conjugated molecule and thiol-gold interactions. Various other determinant-specific binding substance combinations are contemplated for use in practicing the methods of this invention, such as will be apparent to those skilled in the art.

The term “detectably label” is used herein to refer to any substance whose detection or measurement, either directly or indirectly, by physical or chemical means, is indicative of the presence of the target bioentity in the test sample. Representative examples of useful detectable labels, include, but are not limited to the following: molecules or ions directly or indirectly detectable based on light absorbance, fluorescence, reflectance, light scatter, phosphorescence, or luminescence properties; molecules or ions detectable by their radioactive properties; molecules or ions detectable by their nuclear magnetic resonance or paramagnetic properties. Included among the group of molecules indirectly detectable based on light absorbance or fluorescence, for example, are various enzymes which cause appropriate substrates to convert, e.g., from non-light absorbing to light absorbing molecules, or from non-fluorescent to fluorescent molecules.

II. Uses of Abasic DNA Labeling

In accordance with the present invention, abasic DNA labeling is a method which may be used to advantage to study individual nucleic acid molecules. Specifically, abasic DNA labeling may be used to identify AP sites that are placed as landmarks on individual nucleic acid molecules in a sequence dependent manner. By placing AP sites at known positions throughout a DNA molecule, the method may be used to confirm DNA sequence information, as well as evaluate quickly the results of DNA sequencing. Additionally, by combining four sets of data corresponding to abasic DNA molecules lacking A, C, G and T residues, abasic DNA labeling may be used for sequencing DNA molecules.

In a further embodiment of the present invention, abasic DNA labeling may be used to identify AP sites that are naturally formed by ionizing radiation, mutagenic chemicals or spontaneous mutation.

Abasic DNA labeling may also be used for restriction digest mapping on individual DNA molecules. The method may be adapted for mapping protein-DNA interaction sites, and more specifically, abasic DNA labeling may be used for mapping other forms of DNA damage, such as mismatch DNA.

In yet a further embodiment of the present invention, abasic DNA labeling may be applied to characterize methylation patterns from individual DNA molecules. Differential methylation is considered to play a role in development and progression of genetic diseases, but it is difficult to use conventional tools to detect methylation patterns with high resolution. Since the method described here does not require DNA amplification, the in vivo methylation pattern can be determined from a minute amount of DNA sample.

Abasic DNA labeling may be used as a research tool in genetic screening assays to identify those patients that may be at risk for certain genetic disorders. Such disorders include, without limitation, sickle cell anemia, cystic fibrosis, α and β thalassemias, inborn errors of metabolism, familial leukemia, fragile x syndrome, hereditary ataxias, Huntington's disease, Kennedy's disease, myotonic dystrophy, Parkinson's disease, schizophrenia, Ataxia-telangiectasia and Nijmegen breakage syndrome.

Abasic DNA labeling may also be used to advantage to identify gene loci associated with particular genetic disorders as well as for identifying alterations in particular DNA regions which give rise to genetic disorders. Abasic DNA labeling may also be adapted for purposes of identifying infectious bacteria or viruses that have been isolated from infected individuals.

Additionally, the abasic DNA labeling method of the present invention provides the basis for new and improved paternity testing as well as improved means for assessing forensic evidence.

Further details regarding the practice of this invention are set forth in the following examples, which are provided for illustrative purposes only and are in no way intended to limit the invention.

### EXAMPLE I

### Abasic DNA Labeling Using Uracil DNA Glycosylase

An atomic force microscopy-based method for detecting abasic sites (AP sites) on individual DNA molecules which is specific for the removal of thymine (T) residues from individual DNA molecules has been developed in accordance with the present invention. T residues were removed by uracil DNA glycosylase and replaced by uracil (19, 20). Uracil residues were subsequently removed with uracil glycosylase giving rise to AP sites on the treated DNA molecule.

To facilitate atomic force microscopy (AFM) identification of AP sites on individual DNA molecules, AP sites were labeled with biotinylated aldehyde-reactive probes (bio-ARP, 445 Da) and monomeric Avidin (approximately 16 kDa). The predominant reactive group at the AP sites was an open-chain aldehyde derived from the DNA's ribose molecules, and bio-ARP served as an initial marker of the aldehyde group on the AP sites (21, 22). The location of bio-ARPs on the individual DNA molecules was then visualized by AFM via monomeric Avidin bound to bio-ARP.

The above-described procedure was used to incorporate two uracil residues into a 250-bp DNA template. In order to determine the location of AP sites without directional ambiguity, one 5′-end of a single DNA strand was biotinylated and marked with Avidin. The DNA template was gently uncoiled and immobilized on a flat mica surface, and the location of the two Avidin-bound AP sites were identified by AFM at nanometer resolution.

The described AFM-based method facilitates analysis of biochemical reactivity at specific AP sites and can further be used as a means to determine the efficiency of AP-site labeling. AP sites exist in one of three tautomeric forms, such as an open-chain aldehyde, an open-chain hydrate, and hemiacetals, and they can be opposed to any of the four different bases on its complementary strand. In this example, the open-chain aldehyde group was targeted and labeled with bio-ARP and the two AP sites were both opposed to an Adenine residue. Because of tautomerization, any AP site takes the aldehyde form for approximately 1% of time, and therefore, may be labeled by aldehyde-reactive probes (22). Alternatively, there is an enzyme that specifically cleaves the AP site and generates a 5′-end base-free deoxyribose phosphate (23, 24).

I. Materials and Methods

The following protocols are provided to facilitate the practice of the present invention.

Preparation of DNA Templates

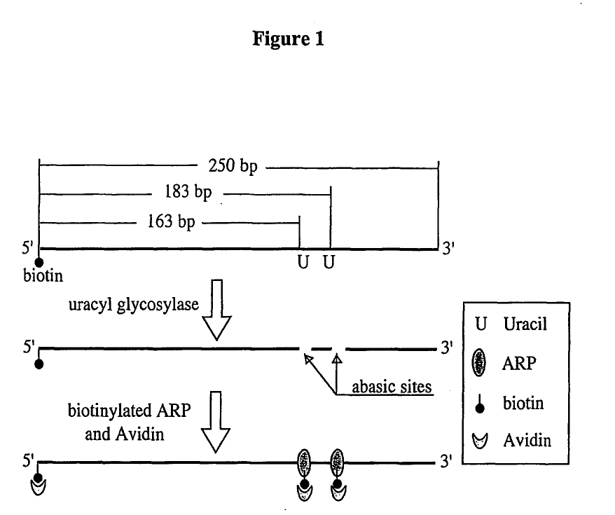

A double-stranded DNA template, 250 bp in length, was constructed which included two uracils located 163 bp and 183 bp away from the biotinylated 5′-end on the upper DNA strand (FIG. 1). The two uracil residues were subsequently removed from the DNA templates using thermolabile uracil N-glycosylase (HU59100, Epicenter Technology). One hundred ng of DNA molecules were incubated for 30 minutes at room temperature in 10 μl of buffer containing 50 mM Tris-HCl (pH 9.0), 20 mM ammonium sulfate, and 1 U of uracil N-glycosylase.

The glycosylated DNA templates containing the two AP sites were then labeled with 5 mM N-aminooxyacetyl-N′-D-biotynoyl hydrazine (bio-ARP, Dojindo Laboratories) in a 10 μl volume of buffer consisting of 10 mM Tris-HCl (pH 8.3), 1.5 mM MgCl₂, and 50 mM KCl for 30 minutes at room temperature. The DNA templates were separated from the uracil N-glycosylase and the unreacted bio-ARP using phenol-chloroform extraction and ethanol precipitation. Lastly, the DNA sample was reacted with monomeric Avidin, approximately 16 kDa in mass (A2036, Sigma), in a buffer containing 50 mM Tris-HCl (pH 8.0), 100 mM NaCl, and 10 mM MgCl₂ for 30 minutes at room temperature. The ratio of Avidin monomers to DNA molecules in the reaction tube was approximately 10.

Uncoiling and Immobilizing DNA Samples

The glycosylated, end-biotinylated DNA templates labeled with bio-ARP and Avidin monomers were gently stretched and immobilized on a freshly cleaved mica surface (25 mm×25 mm, Ted Pella Inc.) by the method previously described (25). Control DNA molecules were also used without glycosylation or end-biotinylation. The mica sheet was then mounted on the custom-made spin-stretcher, and the mica plate was spun at approximately 5000 rpm. A series of solutions were gently dispensed on the spinning center in the order of 50 μl of H₂O, 50 μl of 500 mM MgCl₂, 50 μl of H₂O, 10 μl of the DNA sample, and 50 μl of H₂O in 15 second intervals.

Imaging by Atomic Force Microscopy

A Nanoscope III atomic force microscope (Digital Instruments, Inc.) was used to capture topographical images of monomeric Avidin, DNA templates, and Avidin-DNA complexes immobilized on the mica surface. AFM was operated in the ambient air at 15-20% humidity. The tapping mode was used to reduce any damage to biological samples caused by physical contact with the tip, and the tapping frequency was set to ˜290 kHz. The scanning field of view was 2 μm×2 μm (coarse scanning) or 500 nm×500 nm (fine scanning) with the scanning rate of 0.5-1 Hz and 512 scanning lines. The silicon tips had an estimated curvature of 10 to 20 nm. Height images in the range of 0-5 nm were flattened to remove the background curvature of the mica surface, and the images were analyzed using NIH Image 1.60 image analysis software. The normalized position corresponding to AP sites was defined as d₁/(d₁+d₂), where d₁ and d₂ were the length of DNA segments bisected by a bound Avidin monomer.

II. Results

Labeling AP Sites with Monomeric Avidin

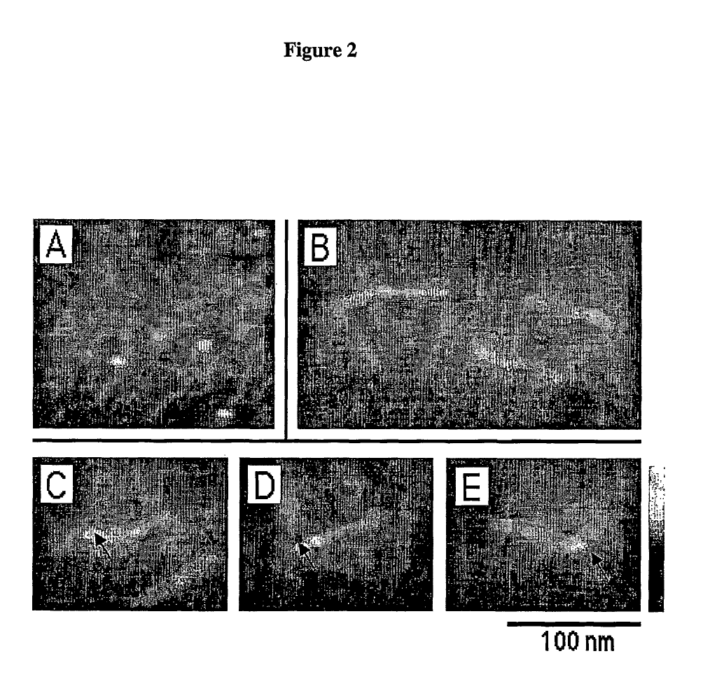

Prior to locating biotin-labeled AP sites on individual DNA molecules, a geometric size of the 250-bp DNA template, as well as monomeric Avidin, was determined, and proper sample preparations were evaluated. In a scanning field of 2 μm×2 μm, approximately 30 DNA molecules were detected, and the mean and the standard deviation of end-to-end DNA length was measured as 77±5 nm (N=45, sample number; FIG. 2). The predicted length in B-form was 82 nm. Monomeric Avidin was elliptical-shaped with the mean and the standard deviation of 16±2 nm (major axis, N=30) and 9±1 nm (minor axis, N=30; FIG. 2). The dimension of a natural form of Avidin consisting of four monomer units was measured as 23±3 (major axis, N=30) and 16±2 (minor axis, N=30)

The monomer form of Avidin was used in this study in order to enhance resolution in determining Avidin's binding sites. When the DNA template was not biotinylated at the 5′-end, approximately 20% of the molecules were detected as a DNA-protein complex (FIG. 2). The binding positions of avidin were in the vicinity of one of the two AP sites at 163 bp and 183 bp from the 5′-end. Approximately 1% of the DNA molecules exhibited two Avidin monomers closely spaced next to each other. For the DNA templates end-labeled with biotin, over 90% of the molecules were end-marked with Avidin when the ratio of DNA molecules to Avidin monomers was set to 10.

Determination of AP Sites

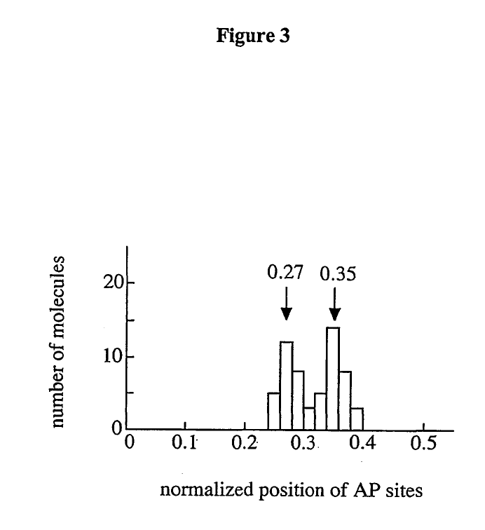

The location of AP sites was first determined using the DNA template lacking the biotinylated 5′-terminus. When straightened, the DNA template had an end-to-end length in the range of 77-84 nm. Since the DNA template was not end-marked, the DNA-bound Avidins could have been on either the segment close to the 5′-terminus or the 3′-terminus. Thus, the binding sites were assigned between 0 and 0.5 in a normalized scale where the end-to-end length of an individual DNA template was set to 1. A histogram of the stretched DNA template was then prepared which exhibited two peaks located at 0.28±0.02 (mean±standard deviation) (N=29) and 0.35±0.02 (N=29; FIG. 3). These observed peaks were in good agreement with the predicted values of 0.27 and 0.35.

Simultaneous Labeling of AP Sites and DNA Ends

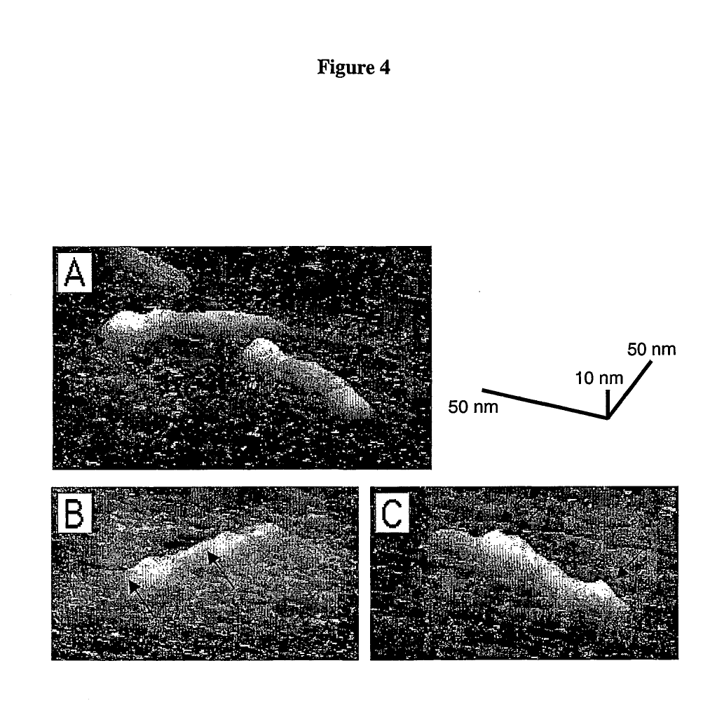

In order to remove directional ambiguity and to distinguish one AP site located on a DNA segment close to the 5′-terminus from the other on the 3′-terminus, biotin was conjugated to the 5′-end of one DNA single strand. Since biotin was incorporated at the 5′-end as well as the two AP sites, all three sites could be simultaneously labeled with Avidin monomers. AFM was able to detect Avidin monomer bound to the DNA end (FIG. 4). The efficiency of DNA end-labeling was approximately 90%, and approximately 25% of the end-labeled DNA molecules exhibited bound Avidin at sites close to the two AP sites. The AFM images clearly showed that the AP sites were located away from the 5′-end marked by Avidin (FIGS. 1 and 4). The observed distance between the two AP sites illustrated in FIG. 4C was 8 nm which was in good agreement with the predicted distance of 7 nm.

III. Discussion

Two AP sites were created and then visualized on a 250-bp DNA template using the rapid and sensitive ARP-mediated method of identifying the position of DNA abasic sites by AFM. The location of the two AP sites were determined from the site of bound Avidins on the uncoiled DNA. The binding of Avidin to the two AP sites, as well as the biotinylated DNA end, was specific in the AFM-based assay. The average difference between the observed AP site and the predicted AP site was within a few nanometers, and the standard deviation of the distribution was approximately 6 bp (˜2 nm) in the observed population of 58 bound Avidins. A pair of Avidins, apparently bound tandemly at the two AP sites separated by 20 bp, was clearly identifiable from a shape resembling a cluster of two globular structures.

Unlike conventional molecular tools, such as electrophoretic mobility shift assays or DNase footprinting, the AFM-based method described herein allows for the analysis of large DNA templates from a minute amount of sample. DNA molecules over 100 kbp in length can be straightened easily by the stretching apparatus previously developed, and determining sites of bound proteins such as Avidin along uncoiled DNA molecules is straightforward. Additionally, by end-labeling the 5′-terminus or the 3′-terminus of a single DNA strand, binding sites of interest can be identified without directional ambiguity.

In conclusion, the abasic DNA labeling method described herein provides a rapid and sensitive tool for identifying AP sites on individual DNA molecules. This method is an improvement over currently available calorimetric or fluorescence-based assays. The mean number of AP sites from a population of DNA molecules are not only quantified, but localized at nanometer resolution.

### EXAMPLE II

### Methylation-Mediated DNA Labeling

AFM may also be implemented for the detection of AP sites on individual DNA molecules which are created by the removal of methylated Cytosine residues (5-methylcytosines). Using this method, individual DNA molecules will be characterized based on the distribution of 5-methylcytosines. Removal of the 5-methylcytosines from the DNA molecule will create AP sites which can be labeled and identified by AFM.

Cytosine (C) residues, recognized at specific recognition sites by methylases, are hemi-methylated. Such recognition sites and methylases include: C in 5′-AGCT-3′ by Alu I methylase, the first C in 5′-GGCC-3′ by Hae III methylase, the C in 5′-GCGC-3′ by HhaI methylase, the second C in 5′-CCGG-3′ by HpaII methylase, the first C in 5′-CCGG-3′ by MspI methylase, and C in 5′-CG-3′ by SssI methylase.

AP sites will be formed by the removal of 5-methylcytosines using 5-methylcytosine-DNA glycosylase. The AP sites will be labeled by biotinylated aldehyde-reactive probes (N-aminooxylacetyl-N′-D-biotynoyl hydrazine) as described in Example 1. Monomeric Avidin molecules may then be reacted with biotin to facilitate detection of AP sites on individual DNA molecules by AFM.

### EXAMPLE III

### Methods for Creating AP Sites Using Various DNA Glycosylases

A family of DNA glycosylases has been identified, and they are known to create AP sites corresponding to specific nucleotides. For instance, 3-methyladenine-DNA glycosylase, detected in humans, is able to remove 3-methyladenine which creates AP sites specific to adenine residues. The same enzyme can remove 3-methylguanine and 7-methylguanine to form AP sites specific to guanine residues. Therefore, it is possible to create AP sites corresponding to any one of the four DNA nucleotides using specific glycosylases. These glycosylases include, but not limited to, uracil-DNA glycosylase, 3-methyladenine-DNA glycosylase, AlkA protein, 5-methylcytosine-DNA glycosylase, Fpg protein, and Tag protein.

Thymine (T) residues may be removed using at least two glycosylases. First, uracil may be incorporated into a target DNA molecule, as described in Example 1, followed by removal of the uracil by uracil-DNA glycosylase. T residues may also be removed by incorporation of O₂-methylthymine into a target DNA molecule, followed by removal of O₂-methylthymine using AlkA protein.

Cytosine (C) residues may be removed using at least three different glycosylases. C residues may be removed by methylating C residues of a target DNA molecule, as described above in Example 2, followed by removal of 5-methylcytosine using 5-methylcytosine-DNA glycosylase. C residues may also be removed by incorporating O₂-methylcytosine into a target DNA molecule, followed by the removal of O₂-methylcytosine using AlkA protein. Lastly, C residues may be removed by incorporating 5-hydroxycytosine into a target DNA molecule, followed by the removal of 5-hydroxycytosine using Fpg protein.

Adenine (A) residues may be removed by incorporating 3-methyladenine into a target DNA molecule, followed by the removal of 3-methyladenine using 3-methyladenine-DNA glycosylase, AlkA protein, or Tag protein.

Guanine (G) residues may be removed using at least three different glycosylases. G residues may be removed by incorporating 3-methylguanine into a target DNA molecule, followed by removal of 3-methylguanine using 3-methyladenine-DNA glycosylase, AlkA protein, or Tag protein. G residues may also be removed by incorporating 7-methylguanine into a target DNA molecule, followed by the removal of 7-methylguanine using 3-methyladenine-DNA glycosylase, or AlkA protein. Lastly, G residues may be removed by incorporating 8-oxoguanine or 7-alkylguanine into a target DNA molecule, followed by removal of 8-oxoguanine or 7-alkylguanine using Fpg protein.

Once the specific nucleotides have been removed using any of the above-described glycosylases, the AP sites may be labeled using biotinylated aldehyde-reactive probes (N-aminooxylacetyl-N′-D-biotynoyl hydrazine) as described in Example 1. Monomeric Avidin molecules may then be reacted with the biotin to facilitate detection of the AP sites on the individual DNA molecules by AFM.

### EXAMPLE IV

### Fluorescence-Based Microscopy Detection of AP Sites

The above described Abasic DNA labeling methods may be adapted to allow for fluorescence-based microscopy detection of AP sites on individual DNA molecules instead of AFM. The conversion may be accomplished by conjugating fluorescent dye to the biotinylated probes which will facilitate the detection and characterization of DNA molecules using fluorescence microscopy.

The fluorescent labeled probes have additional applications, including the creation of digital DNA images unique to individual DNA molecules. Digital images may then be used to identify genetic disorders or heterogeneity among populations.

### References

1. Collins, F. S.; Patrinos, A.; Jordan, E.; Chakravarti, A.; Gesteland, R.; Walters, L. Members of the DOE and NIH planning groups. Science 1998, 282, 682-689.

2. Marshall, E. Science 1999, 284, 1439-1440.

3. Lhomme, J.; Constant, J. F.; Demeunynck, M. Biopolymers 1999, 52, 65-83.

4. Beger, R. D.; Bolton, P. H. J. Biol. Chem. 1998, 273, 15565-15573.

5. Rossi, O.; Carrozzino, F.; Cappelli, E.; Carli, F.; Frosina, G. Int. J. Cancer 2000, 85, 21-26.

6. Binnig, G.; Quate, C. F.; Gerber C. Physical Rev. Let. 1986, 56, 930-933.

7. Hansma, P. K.; Elings, V. B.; Marti, O.; Bracker, C. E. Science 1988, 242, 209-242.

8. Bustamante, C.; Vesenka, J.; Tang, C. L.; Rees, W.; Guthold, M.; Keller, R. Biochem. 1992, 31, 22-26.

9. Hansma, H. G.; Laney, D. E.; Bezanilla, M.; Sinsheimer, R. L.; Hansma, P. K. Biophys. J. 1995, 68, 1672-1677.

10. Radmacher, M.; Fritz, M; Hansma, H. G.; Hansma, P. K. Science 1994, 265, 1577-1579.

11. Guthold, M.; Bezanilla, M.; Erie, D. A.; Jenkins, B.; Hansma, H. G.; Bustamante, C. Proc. Natl. Acad. Sci. 1994, 91, 12927-12931.

12. Sun, H. B.; Yokota, H. Anal. Chem. 2000, 72, 3138-3141.

13. Kubo, K.; Ide, H.; Wallace, S. S.; Kow, Y. W. Biochem. 1992, 31, 3703-3708.

14. Makrigiorgos, G. M.; Chakrabarti, S.; Mahmood, A. Int. J. Radiat. Biol. 1998, 74, 99-109.

15. Nakamura, J.; Walker, V. E.; Upton, P. B.; Chiang, S. Y.; Kow, Y. W.; Swenberg, J. A. Cancer Res. 1998, 58, 222-225.

16. Demple, B.; Harrison, L. Annu. Rev. Biochem. 1994, 63, 915-948.

17. Cadet, J.; Berger, M.; Douki, T.; Morin, B.; Raoul, S.; Ravanat, J. L.; Spinelli, S. Biol. Chem. 1997, 378, 1275-1286.

18. Beckman, K. B.; Ames, B. N. J. Biol. Chem. 1997, 272, 19633-19636.

19. Hayakawa, H.; Kumura, K.; Sekiguchi, M. J. Biochem. 1978, 84, 1155-1164.

20. McCullough, A. K.; Dodson, M. L.; Lloid, R. S. Annu. Rev. Biochem. 1999, 68, 255-285.

21. Monoharan, M.; Ransom, S. C.; Mazumder, A.; Gerlt, J. A. J. Am. Chem. Soc. 1988, 110, 1620-1622.

22. Doetsch, P. W.; Cunningham, R. P. Mutation Res. 1990, 236, 173-201.

23. Srivastava, D. K., Vande Berg, B. J.; Prasad, R.; Molina, J. T.; Beard, W. A.; Tomkinson, A. E.; Wilson, S. H. J. Biol. Chem. 1998, 273, 21203-21209.

24. Krokan, H. E.; Nilsen, H.; Skorpen, F.; Otterlei, M.; Slupphaug, G. FEBS Lett. 2000, 476, 73-77.

25. Yokota, H.; Sunwoo, J.; Sarikaya, M.; van den Engh, G.; Aebersold, R. Anal. Chem. 1999, 71, 4418-4422.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for characterizing individual nucleic acid molecules, comprising the steps of:
a) forming abasic sites specific for at least one nucleic acid base on individual nucleic acid molecules,
b) labeling said abasic sites with detectably labeled molecules, and
c) detecting the location of said detectably labeled molecules on said individual nucleic acid molecules, thereby determining the nature and location of said abasic sites.

2. The method of claim 1, wherein said nucleic acid molecules are selected from the group consisting of cDNA and DNA.

3. The method of claim 1, wherein said abasic sites are created using a glycosylase selected from the group consisting of uracil-DNA glycosylase, 3-methyladenine-DNA glycosylase, AlkA protein, 5-methylcytosine-DNA glycosylase, Fpg protein, and Tag protein.

4. The method of claim 1, wherein atomic force microscopy is performed for said detection.

5. The method of claim 1, wherein fluorescence-based microscopy is performed for said detection.

6. A method for characterizing individual nucleic acid molecules as claimed in claim 1, wherein said abasic sites formed in step a) results from removal of A residues, said method further comprising:
d) providing three additional identical nucleic acid molecules, where each of said three nucleic acid molecules are treated to remove C, G and T residues, respectively, thereby generating four nucleic acid sequences, each of said four nucleic acid sequences containing abasic sites corresponding to a particular nucleic acid base, and
e) determining the location of each abasic site in said four nucleic acid molecules, thereby determining the sequence of said nucleic acid molecule.

7. The method of claim 1, wherein said method is used to identify abasic sites that are naturally formed in vivo by a method selected from the group consisting of ionizing radiation, mutagenic chemicals and spontaneous mutations.

8. The method of claim 1, wherein said method is used to identify patients in need thereof who are at risk for certain genetic disorders including those genetic disorders selected from the group consisting of sickle cell anemia, cystic fibrosis, α and β thalassemias, inborn errors of metabolism, familial leukemia, fragile X syndrome, hereditary ataxias, Huntington's disease, Kennedy's disease, myotonic dystrophy, Parkinson's disease, schizophrenia, Ataxia-telangiectasia and Nijmegen breakage syndrome.

9. The method of claim 1, wherein said method is used to identify infectious bacteria or viruses present in a biological sample.

10. The method of claim 1, wherein said method is used to determine paternity of an individual.
